(19) United States
(12) Patent Application Publication
(10) Pub. No.: US 2012/0048278 A1
(43) Pub. Date: Mar. 1, 2012

(54) OROPHARYNGEAL AND NASOPHARYNGEAL AIRWAY HAVING A BREATHING INDICATOR

(76) Inventor: Anthony John Yasick, West Olive, MI (US)

(21) Appl. No.: 13/214,637

(22) Filed: Aug. 22, 2011

Related U.S. Application Data
(60) Provisional application No. 61/376,340, filed on Aug. 24, 2010.

(51) Int. Cl. A61M 16/04; A61M 16/00

(52) U.S. Cl. 128/207.14; 128/207.18

(57) ABSTRACT

An oropharyngeal airway configured for placement within a mouth of a patient to create a passageway between a mouth of a patient and the posterior pharyngeal wall that includes a passageway body, a flange assembly and a reactive material. The flange assembly is positioned at the end of the body and includes spaced apart upper and lower flange portions, which define a passageway therebetween which is in communication with the opening of the passageway body. The reactive material cooperates with the flange assembly and the passageway defined by the spaced apart flange portions. The reactive material provides qualitative visual indication as to whether the patient is effectively ventilating. A nasopharyngeal airway is likewise disclosed, as is a flange assembly which can be coupled to existing airways.

### CROSS-REFERENCE TO RELATED APPLICATION

The present disclosure claims priority from U.S. Prov. Pat. App. Ser. No. 61/376, 340 filed Aug. 24, 2010, entitled “Oropharyngeal and Nasopharyngeal Airway Having an Indicator.” The entire disclosure of the provisional application is hereby incorporated by reference.

### BACKGROUND OF THE DISCLOSURE

1. Field of the Disclosure

The disclosure relates in general to airway adjuncts for maintaining an open airway of a patient, and more particularly, to an oropharyngeal or a nasopharyngeal airway having an indicator to confirm that the patient is effectively ventilating (also commonly referred to as effectively breathing), in a self-contained unit, even from a distance, and without difficulty.

2. Background Art

The use of a various airway adjuncts is well known in the art. Amongst a number of others, it is common to utilize either one of an oropharyngeal or a nasopharyngeal airway, especially in patients who are unconscious or sedated to various degrees. These airways assist the patient in keeping an open airway to facilitate respiration.

Typically, a oropharyngeal airway comprises an elongated and generally curved polymer tube that is inserted into the mouth of the user and over the tongue. The oropharyngeal airway, once positioned, creates an air passageway between the mouth and the posterior pharyngeal wall. This is quite useful when the tongue and/or the epiglottis fall backwards against the posterior pharynx in patients that are anesthetized or unconscious.

A nasopharyngeal airway is similar to the oropharyngeal airway except that it is extended through the nostril to create a passageway between the nose and the nasopharynx. It is usually a bit longer than the oral airway.

Once installed, the patient may not necessarily be monitored as closely with respect to the continued operation of the device. Thus, certain problems can result. Even in closely monitored patients, it can be difficult to ascertain as to whether the person is effectively breathing. Also, in some care settings (i.e.: recovery room) patients aren't monitored on a one-on-one basis. Often times, breathing problems may not be noticed immediately. Obviously, if breathing issues persist, even for a short period of time, severe injury or death can result.

A variety of products have been developed that provide monitoring of, for example, carbon dioxide, levels in the breath of a patient. Such devices, many of which are available under the brand name Portex® are available from Smiths Medical, of Ashford, Kent, United Kingdom, and of Dublin, Ohio. Typically such devices comprise in-line devices that are coupled to ventilators and endotracheal tubes. One example comprises a tubular member with a flat panel exiting tangentially from the tubular member. The tangential member includes a carbon dioxide sensor. Other sensors are placed in equally large and cumbersome equipment that is generally configured to function with in-line devices.

With such equipment, the respiratory passageways of the user are coupled to larger devices that are positioned remote from the user. As such, while there are some concerns as to size, typically, space is not at a premium, and there is ample space around which to place the detector.

However, unlike a ventilators and endotracheal tubes, or other resuscitators, an airway is designed to terminate at the opening of the mouth (or the nose in the case of a nasopharyngeal airway). This is because it is often the case that the mouth and the nose may be covered with a mask, a breathing apparatus or other apparatus. For example, during anesthesia, masks or ventilator circuits may include equipment that is positioned over the nose and mouth of a patient. So that there is no inadvertent blocking, hitting or dislodging of the airway, or impairment of the function of the airway, the less that the airway extends past the nasal or mouth cavity, the less risk that is present. Thus, the devices of the prior art are not suitable for use with airways, and would be difficult if not impossible to couple to an airway without degrading the performance of the airway.

Thus, it is an object of the present disclosure to provide an airway which has an indicator that can provide instant feedback as to the breathing pattern of the user.

It is another object of the present disclosure to provide fast, reliable and easy visualization of the breathing pattern of a patient.

It is another object of the disclosure to provide an indicator as to whether or not the patient is breathing effectively.

These objects as well as other objects of the present disclosure will become apparent in light of the present specification, claims, and drawings.

### SUMMARY OF THE DISCLOSURE

The disclosure is directed to oropharyngeal and nasopharyngeal airways which provide indication relative to respiration with minimal intrusion and with ease of visibility by a practitioner. As will be understood, the disclosure is not limited to the configuration of any particular airway, and is not limited to Guedel airways in particular.

More specifically, the disclosure is directed to an oropharyngeal airway configured for placement within a mouth of a patient to create a passageway between a mouth of a patient and the posterior pharyngeal wall. The airway includes a passageway body, a flange assembly, and a reactive material. The passageway body includes a proximal end and a distal end. The passageway body defines a passageway extending therethrough with an opening at the proximal end and an opening at the distal end. It will be understood that such a passageway may comprise an elongated slot, or elongated tubular member, among other structures.

The flange assembly is positioned at the proximal end of the passageway body. The flange assembly is configured to reside proximate a mouth of a patient when in use. The flange assembly includes an upper flange portion and a lower flange portion, which are spaced apart from each other to define a passageway therebetween and in fluid communication with the proximal end of the passageway body. In such a configuration, at least a portion of the air extending through the passageway body is directed through the passageway defined by the spaced apart flange portions.

The reactive material cooperates with the flange assembly with the reactive material in communication with the passageway defined by the spaced apart flange portions. The reactive material provides a qualitative visual indication as to whether the patient is effectively ventilating.

In some preferred embodiments, the reactive material is positioned on a lower surface of the upper flange portion so that it lies directly across from the opening of the passageway at the proximal end of the passageway body, and so that it is visible through the upper flange portion. For example, the upper flange portion can be made of transparent, semi-transparent, or translucent material or may include an opening that allows for visualization of the reactive material therebelow.

In some preferred embodiment, at least a portion of the upper flange portion is substantially perpendicular to an axis defined by the passageway of the passageway body near the proximal end.

In some such preferred embodiments, the upper flange portion is substantially parallel to the lower flange portion and spaced apart less than one inch therefrom. It is desirable that the two are spaced apart even less, for example, than one half inch, or even less.

In other preferred embodiments, an indicator sticker is positioned on the upper flange portion proximate the reactive material, to, in turn, allow for comparison thereto.

In other preferred embodiments, the area of the passageway defined by the upper and lower flange portions is at least 80% the area of the passageway at the opening at the proximal end of the passageway body. Such a configuration is thought to allow contact with the reactive material while minimizing any obstruction to the flow of air.

In some preferred embodiments, the upper flange further includes an opening extending therethrough. The opening is positioned above the opening at the proximal end of the passageway body. It is sized so as to be smaller than the opening at the proximal end of the passageway body.

In some preferred embodiments, the upper flange portion and the lower flange portion are coupled to each other through a plurality of spacers that, in turn, define the spacing of the passageway therebetween.

In other embodiments, the upper flange, the lower flange portion and the plurality of spacers are integrally molded with each other.

In some embodiments, the airway further includes a bite protector inserted into the opening of the passageway body at the proximal end thereof. The upper flange portion is coupled to the body. Upon insertion of the bite protector and coupling of the upper flange portion thereto, the upper flange portion is in operable position overlying the lower flange portion, while being spaced therefrom. Thus, the upper flange portion and the bite protector may comprise two separate members, or one integrally formed member which can be coupled to existing airway configurations, without modification.

In some other embodiments, the bite protector is integrally molded with the upper flange portion.

In other embodiments, the upper flange portion is spaced apart from the lower flange portion less than half of an inch.

In some embodiments, the upper flange portion defines a footprint and the lower flange portion defines a footprint. The upper flange portion footprint remains within the confines of the footprint of the lower flange portion. In certain configurations, the two flange portions are substantially identically designed, whereas in other configurations, the upper flange portion is smaller than the lower flange portion. It will be understood that the upper flange portion may, in certain embodiments, be larger than the lower flange portion.

It will be understood that the airway may comprise either one of a Berman type or a Guedel type airway, among other specialized or conventional airways.

In another aspect of the disclosure, the assembly is configured for coupling to existing airways. For example, many conventional existing airways have bite protectors which are inserted into the body of the airway to correspond to the position of the teeth in use. The bite protector adds rigidity to the airway at that portion and keeps the airway open. The bite protector, can be configured as an attachment point for the assembly which includes the flange and the reactive material. Alternatively, the flange of an existing airway can itself be utilized to retain the assembly in operable position. In still other embodiments, the assembly can be attached to a passageway body in various different manners. Indeed, there is no limitation as to how the assembly is attached to an existing airway. In such configurations, a flange assembly is couplable to the existing airway. The flange assembly is configured to reside proximate a mouth of a patient when the bite protector is in operable position, and includes an upper flange portion being spaced apart from a proximal end of a passageway body the airway to define a passageway therebetween. At least a portion of the air extending through a passageway body is directed through the passageway defined by the spaced apart flange portion. The reactive material is placed so that it is in the passageway so as to provide a qualitative visual indication as to whether the patient is effectively ventilating.

In certain embodiments, a lower flange portion is spaced apart from the upper flange portion and configured to be positioned proximate a proximal end of a passageway body when the bite protector is properly positioned.

In another aspect of the disclosure, the airway is configured for placement through a nostril of a patient to create a passageway between a nostril of a patient and the posterior pharyngeal wall. The configuration includes a passageway body, a flange assembly and a reactive material. The passageway body has a proximal end and a distal end. The passageway body defines a passageway extending therethrough with an opening at the proximal end and an opening at the distal end.

The flange assembly is positioned at the proximal end of the passageway body. The flange assembly configured to reside proximate a nostril opening of a patient when in use, and, including an upper flange portion and a lower flange portion. The upper and lower flange portions are spaced apart from each other to define a passageway therebetween which is in fluid communication with the proximal end of the passageway body. In such a configuration at least a portion of the air extending through the passageway body is directed through the passageway defined by the spaced apart flange portions.

The reactive material cooperates with the flange assembly and is in communication with the passageway defined by the spaced apart flange portions. The reactive material provides a qualitative visual indication as to whether the patient is effectively ventillating.

In a preferred embodiment, the reactive material is positioned on a lower surface of the upper flange portion so that it lies directly across from the opening of the passageway at the proximal end of the passageway body. In such an orientation, it is configured to be visible through the upper flange portion.

In another embodiment, at least a portion of the upper flange portion is substantially perpendicular to an axis defined by the passageway of the passageway body near the proximal end.

In some such embodiments, the upper flange portion is substantially parallel to the lower flange portion and spaced apart less than one inch therefrom.

In some such embodiments, the upper flange portion and the lower flange portion are coupled to each other through a plurality of spacers. In turn, such a configuration of the spacers define the passageway therebetween.

In some embodiments, an indicator sticker is positioned on the upper flange portion proximate the reactive material, to, in turn, allow for comparison thereto.

### BRIEF DESCRIPTION OF THE DRAWINGS

The disclosure will now be described with reference to the drawings wherein:

FIG. 1 of the drawings is a perspective view of the oropharyngeal airway of the present disclosure;

FIG. 2 of the drawings is a side elevational view of the oropharyngeal airway of the present disclosure;

FIG. 3 of the drawings is a back plan view of the oropharyngeal airway of the present disclosure;

FIG. 4 of the drawings is a partial cross-sectional view of the oropharyngeal airway of the present disclosure, showing, in particular, the flange assembly;

FIG. 5 of the drawings is a partial cross-sectional view of the oropharyngeal airway of the present disclosure, showing, in particular, the lower flange portion and passageways;

FIG. 6 of the drawings is an enlarged cross-sectional view of the oropharyngeal airway of the present disclosure, showing, in particular, the flange assembly;

FIG. 7 of the drawings is a perspective view of an oropharyngeal airway of the present disclosure, showing, in particular, a configuration incorporating a bite protector and an upper flange portion coupled to the bite protector;

FIG. 8 of the drawings is a side elevational view of the oropharyngeal airway of the present disclosure, showing, in particular, a configuration incorporating a bite protector and an upper flange portion coupled to the bite protector;

FIG. 9 of the drawings is a bottom plan view of the oropharyngeal airway of the present disclosure, showing, in particular, a configuration incorporating a bite protector and an upper flange portion coupled to the bite protector;

FIG. 10 of the drawings is a front plan view of the oropharyngeal airway of the present disclosure, showing, in particular, a configuration incorporating a bite protector and an upper flange portion coupled to the bite protector;

FIG. 11 of the drawings is a side elevational view of another embodiment of the oropharyngeal airway of the present disclosure, showing, in particular, a protective shield between the opening of the passageway body and the upper flange portion, to limit the passage of body fluids into contact with the reactive material;

FIG. 12 of the drawings is a perspective view of the nasopharyngeal airway of the present disclosure; and

FIG. 13 of the drawings is a side elevational view of the nasopharyngeal airway of the present disclosure.

### DETAILED DESCRIPTION OF THE DISCLOSURE

While this disclosure is susceptible of embodiment in many different forms, there is shown in the drawings and described herein in detail a specific embodiment with the understanding that the present disclosure is to be considered as an exemplification and is not intended to be limited to the embodiment illustrated.

It will be understood that like or analogous elements and/or components, referred to herein, may be identified throughout the drawings by like reference characters. In addition, it will be understood that the drawings are merely schematic representations of the disclosure, and some of the components may have been distorted from actual scale for purposes of pictorial clarity.

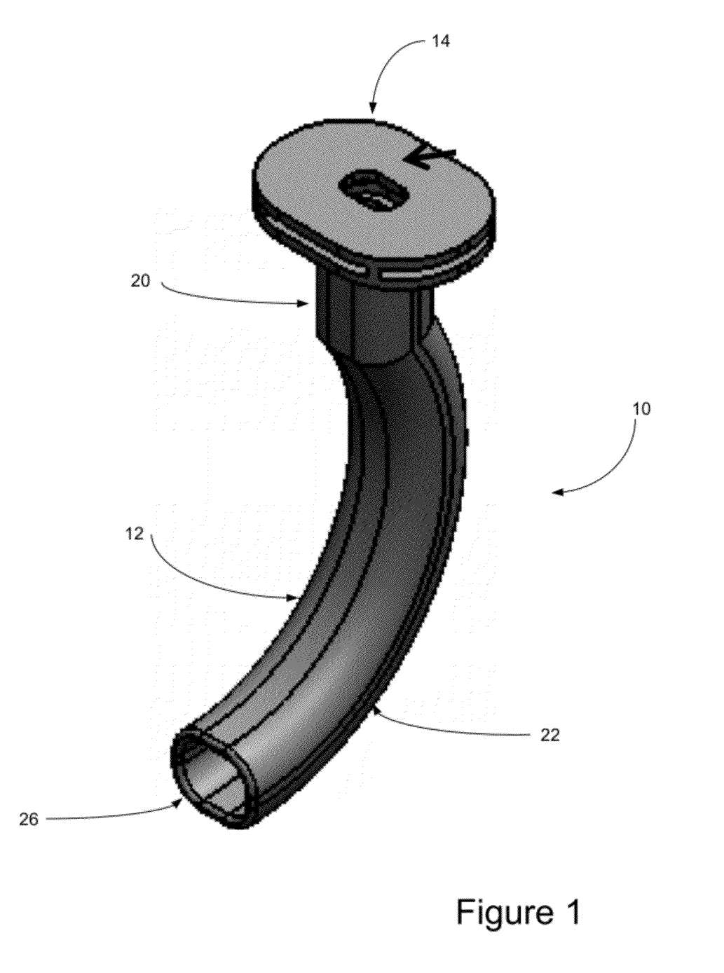

Referring now to the drawings and in particular to FIG. 1, an oropharyngeal airway is shown generally at 10. Typically, such airways come in a number of differently sized and shaped airways so as to accommodate the anatomical features of any number of differently sized individuals. In some instances, a kit may be provided that includes several differently dimensioned oropharyngeal airways. While not required, many such airways are formed from a single molded component (i.e., monolithic). Of course, in other embodiments, the airway can be formed from multiple components that are in some manner joined together. Certain such airways may include bite protectors and the like. It is contemplated, and shown in the drawings, a Guedel type oropharyngeal airway with the understanding that the principles of the disclosure, and the claims associated therewith are equally applicable to Berman type oropharyngeal airways, among other known oropharyngeal airways, without limitation.

The oropharyngeal airway 10 includes passageway body 12 and flange assembly 14. The passageway 12 includes upper straight portion 20, lower curved portion 22, proximal opening 24 (FIG. 6) at the end of the upper straight portion 20 and distal opening 26 at the end of the lower curved portion 22. The passageway body has a substantially uniform thickness and cross-sectional configuration so as to define an elongated passageway through which the patient can breathe. Of course, there can be variations in the cross-sectional configuration of the elongated passageway. For example, the Berman type oropharyngeal airways typically define an i-beam structure which defines two side-by-side passageways segments or portions. The disclosure is not limited to any number of separate passageway segments or portions, as these likewise define a passageway under the present disclosure.

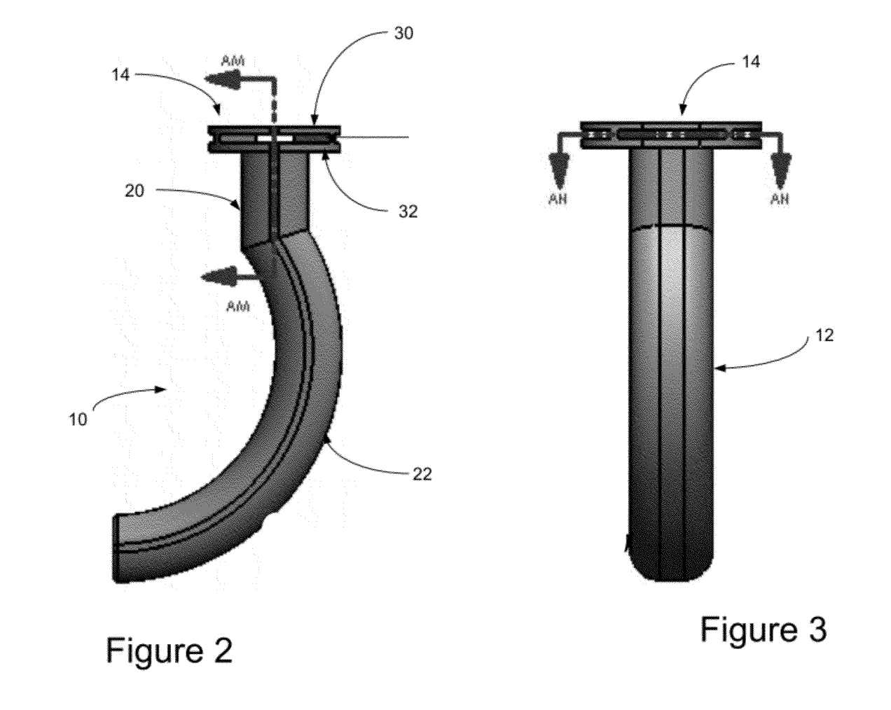

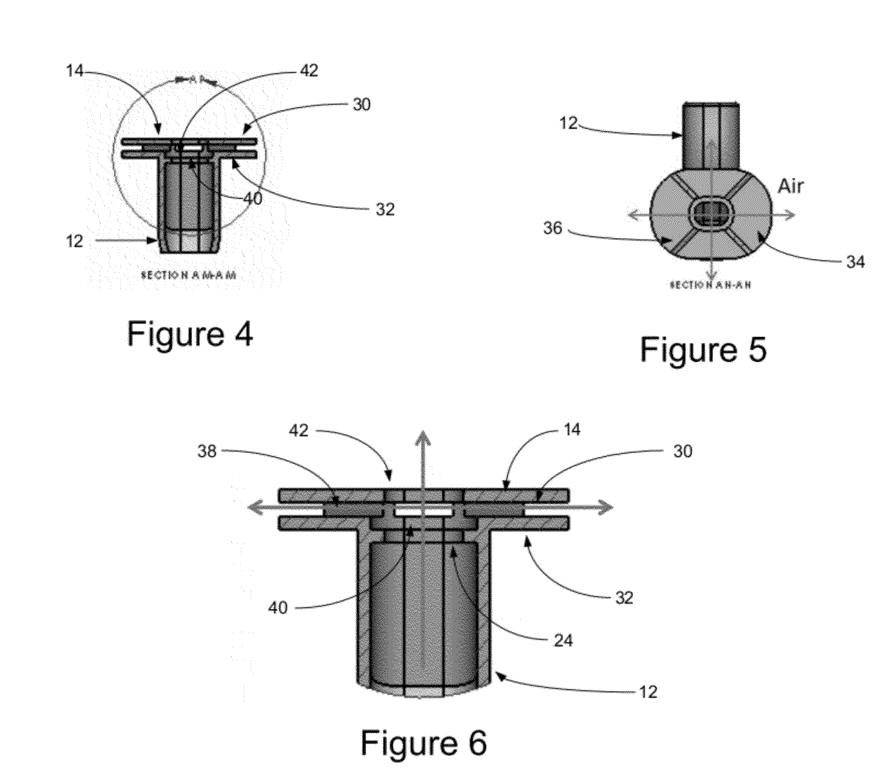

The flange assembly 14 is shown in FIGS. 2 through 6 as comprising an upper flange portion 30, lower flange portion 32, spacers/diverters 36 and reactive material 38. The flange assembly 14 is integrally molded with the passageway body, although a separate configuration is likewise contemplated. The flange assembly is configured to not only aid in the positioning of the airway, but also, to allow a practitioner to see if the patient is breathing, with ease, and without necessarily stopping other tasks.

In particular, the lower flange portion is positioned substantially orthogonal to the upper straight portion 20 of the passageway body 12. Generally, the lower surface of the lower flange portion maintains contact with the lips or mouth of the user when properly positioned. The flange portion is sized so that it does not enter into the mouth, and instead rests on the outer surface of the face or teeth. The lower flange portion includes opening 40 which provides fluid communication with the proximal opening 24.

The upper flange portion 30 is positioned substantially parallel to the lower flange portion 32. The upper and lower flange portions are substantially identical in size, and the upper flange portion includes primary opening 42 which substantially corresponds to the opening 40 of the lower flange portion 32. In the embodiment shown, the primary opening 42 is larger than the opening 40 of the lower flange portion. Thus, and as will be explained, the different sizing of the openings further assists in diverting flow of air between the two flange portions. It will be understood that in other embodiments, the primary opening 42 can be eliminated so as to direct the entirety of airflow along the reactive material 38 and through the side passages 36. It will be understood that the side passages 36 can be sized in such a manner so as to handle the flow of through the lower opening 40 without placing a large restriction thereon. It is further contemplated that the upper flange portion is configured to be the same size or smaller than the lower flange portion so as to minimize the intrusiveness of the upper flange portion. In certain embodiments, it may be larger, however, it is preferred that it remain smaller. In some embodiments, the upper flange portion may be only a fraction of the size of the lower flange portion, to again accommodate a minimal footprint and intrusiveness.

The upper and lower flange portions are spaced apart a predetermined distance, and spacers/diverters 36 maintain the separation and location of the two flange portions relative to each other. The spacers/diverters 36 (hereinafter spacers for simplicity) are radially spaced so as to extend radially outward from the primary opening 42. In the embodiment shown, the spacers are positioned to extend radially in a substantially evenly spaced orientation. Of course, other means by which to separate the two flange portions are contemplated, and other configurations of the spacers are likewise contemplated. Additionally, the spacers need not extend to the outer extents of the various flanges. It will be understood that in other embodiments, the spacers can be placed around the lower opening so as to direct air through a single passageway 34 corresponding to approximately 90° of the flange portion. Of course, the disclosure is not limited to any particular configuration of the flange portions, and the number or placement of the passageways.

It is likewise contemplated that the different flange portions may be something other than parallel to each other. For example, the upper flange portion and/or the lower flange portion may be partially outwardly concave or convex. It will be understood however that the upper flange portion is positioned so that it is substantially perpendicular (or largely perpendicular) to the passageway body and that it is substantially parallel (or largely parallel) to the mouth opening so that the upper flange portion (and in turn, the reactive material) faces directly outward and is easy to see by the medical practitioner. It is contemplated that other orientations may be possible wherein the practitioner can adequately and easily see the status of the patient's respiration by easily seeing the reactive material.

Additionally, it is contemplated that the distance between the two flange portions can be varied so as to achieve different flow patterns between the two flange portions. It is contemplated that, however, there is as little space between the two flange portions as practicable for multiple reasons. First, the further that the reactive material 38 sits from the opening 40, the less respiratory air that makes direct contact with the reactive material 38. Second, the further apart the two flange portions are positioned, the more likely that the upper flange portion 30 and the spacers/diverters 36 will inadvertently come into contact with other equipment, or may be hit by a practitioner, again inadvertently. However, it will be understood that if the upper flange portion is too close, then it may block or otherwise obstruct or negatively impact the respiration of the user. Thus, it is greatly preferred that the upper flange portion remain one inch or less away from the lower flange portion and more preferably, less than a half of an inch away from the lower flange portion. It is desirable to have the upper flange portion as close to the lower flange portion as possible without undesirably obstructing the respiration of the patient. It is preferred that the passageway opening defined by the spaced apart flange portions is at least 80% the area of the opening, although other variations are contemplated, and the 80% is not to be taken as corresponding to the lower limit for undesirable obstruction of the patient's respiration. It will be understood that tests can be conducted to determine the ultimate and optimal placement of the upper flange portion relative to the lower flange portion.

It will be understood that in certain embodiments, the upper and lower flange portions may be formed as a single monolithic member, or a co-molded component. In other embodiments, the upper flange portion can be adhered to or otherwise joined to the lower flange portion. It will be understood that the upper and lower flange portions along with the passageway body comprise a single integrally molded member.

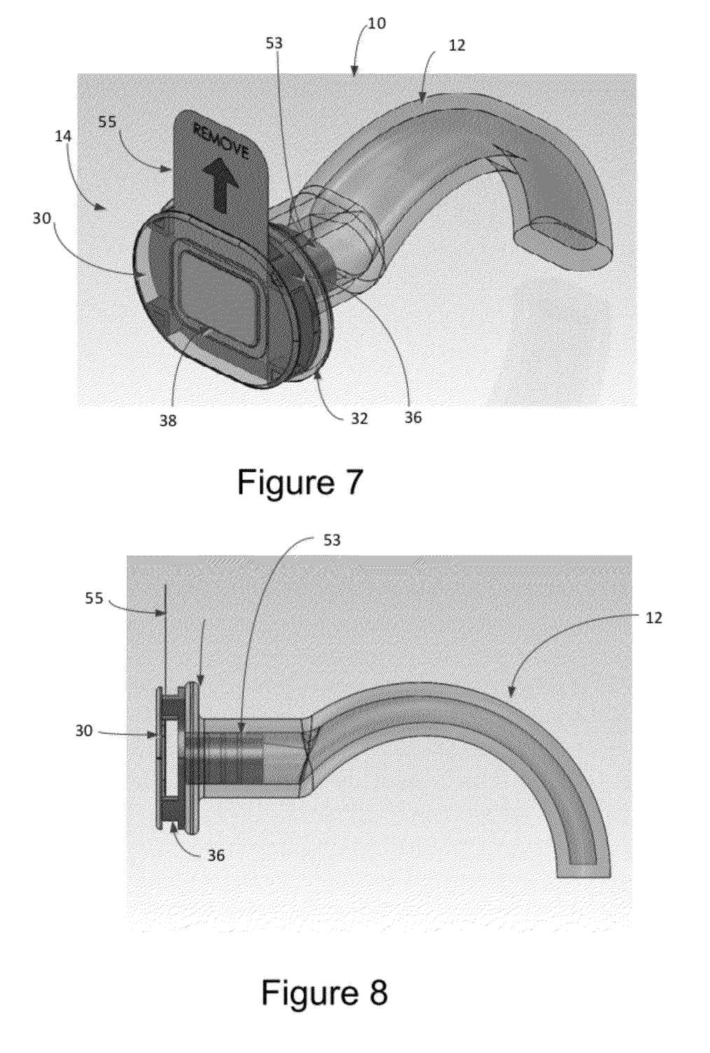

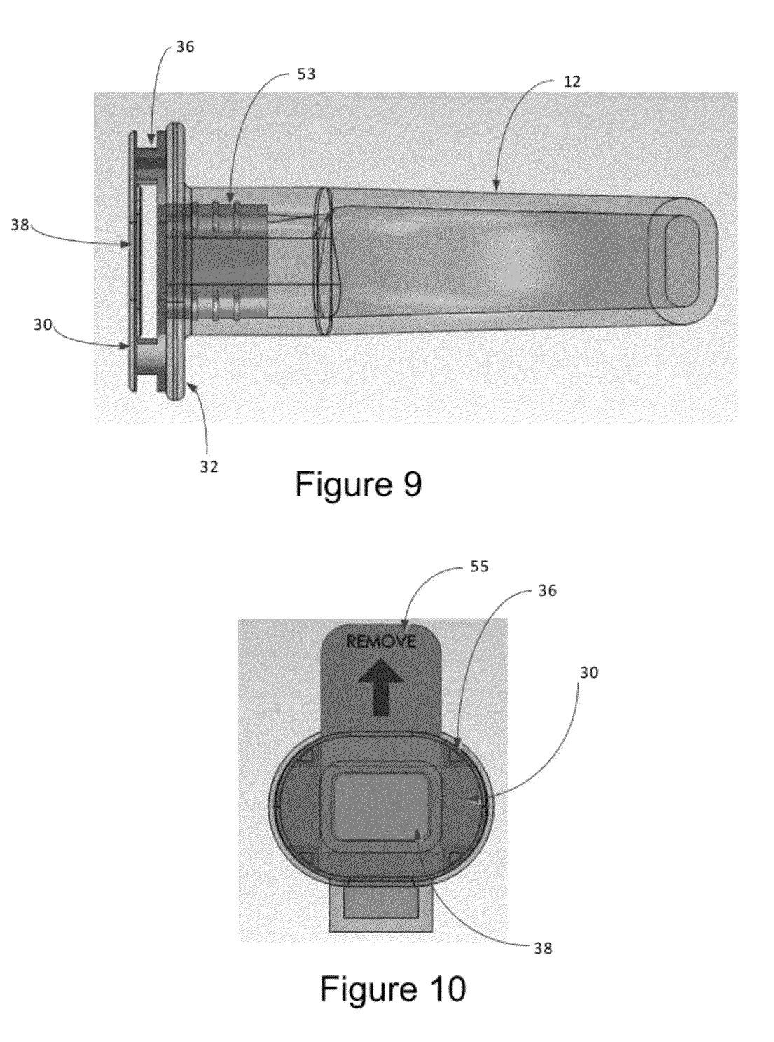

In another embodiment, such as the embodiment of FIGS. 7 through 10, the upper flange portion can be releasably coupled to the lower flange portion or the passageway body. For example, in the embodiment shown, due to the flexibility of the passageway body, the upper straight portion of the passageway body may include a bite protector 53 that is inserted into the straight portion. This more resilient material precludes the collapse of the passageway body through an inadvertent biting by a patient when the device is in use. Such a bite protector is known in the art. However, in the embodiment shown, the upper flange portion 30 can be coupled to the bite protector which is co-molded with spacers 36, such that when the bite protector 53 is inserted into and retained within the upper straight portion in the proper position, the upper flange portion is likewise disposed above the lower opening 40 in the proper position. In other such embodiments, the member can be integrated with the bite protector, or co-molded therewith.

The reactive material 38 is either coated on the surfaces of the flange portions or molded into the flange portions. Most preferably, the reactive material is coated on the lower surface of the upper flange portion and/or the upper surface of the lower flange portion. The reactive material 38 has a visible reaction to a component that is in the air passing through the passageway, to, in turn, identify if the patient is effectively ventilating. In the embodiment contemplated, the reactive material changes color in the presence of carbon dioxide. Among other solutions, certain detectors are shown in U.S. Pub. No. 2008/0081003 published to Ostrowski et al. and U.S. Pub. No. 2006/0216828 published to Ratner et al., the entire disclosures of both of which are hereby incorporated by reference.

Thus, when the patient is inhaling, there is very little noticeable color change, as the user is taking in mostly oxygen and nitrogen. However, when exhaling, there is a large component of carbon dioxide that is being removed from the body. Thus, during the exhaling, the reactive material changes color, and indicates that the patient is (or just did) exhale. As soon as the patient begins the next inhaling step, the reactive material is no longer in the presence of carbon dioxide, the color change is reversed. In the embodiment shown, the material is generally clear (or a first color) in the presence of little to no carbon dioxide, and a highly visible color (or significantly different color than the first color) in the presence of carbon dioxide.

For example, however, if the patient is not effectively ventilating, there will not be a color change in the reactive material. Within a few short moments, a practitioner can easily spot that no color change is occurring, and can react accordingly. Thus, without necessarily needing to see changes in any other vital signs, the practitioner can easily tell whether or not the patient is breathing sufficiently.

Another such reactive material can be found used in association with Portex® branded carbon dioxide detectors, and such reactive material can be applied to the present disclosure. One particular example of reactive material (but not the structure of the present airway) can be seen in the Portex® CO2 Clip Carbon Dioxide Detector No. 001-631125 which includes a color indicator. Such an indicator is incorporated by reference herein. The indicator is blue when there is no carbon dioxide present, green when there is approximately 1-2% carbon dioxide present, green-yellow when approximately 2-5% carbon dioxide is present and yellow when more than 5% carbon dioxide is present. Surrounding the indicator is a sticker that has the different colors printed thereon so that the practitioner can easily and quickly compare the color of the indicator to the color of the sticker. In turn, the practitioner can easily and quickly determine the respiration of the patient.

Such an indicator is comparatively thin and can be adhered or applied to the inside surface of the upper flange portion 30. The comparative sticker can likewise be coupled to the inside or outside of the upper flange portion 30. Of course, this presumes that at least a portion of the upper flange portion is transparent or translucent so that items positioned on the inside surface of the upper flange portion can be readily readable through the flange portion itself It will be understood that in certain embodiments, the comparative sticker can be omitted altogether or may be positioned on the upper surface of the lower flange portion 30. It will be understood a removable seal layer 55 may be positioned over the reactive material, as the reactive material, in certain configurations is activated by removal of the seal layer, and remains active for a predetermined period of time (i.e., 24 hours or the like). Other materials may be utilized which do not require a protective seal layer, or that may last for different periods of time.

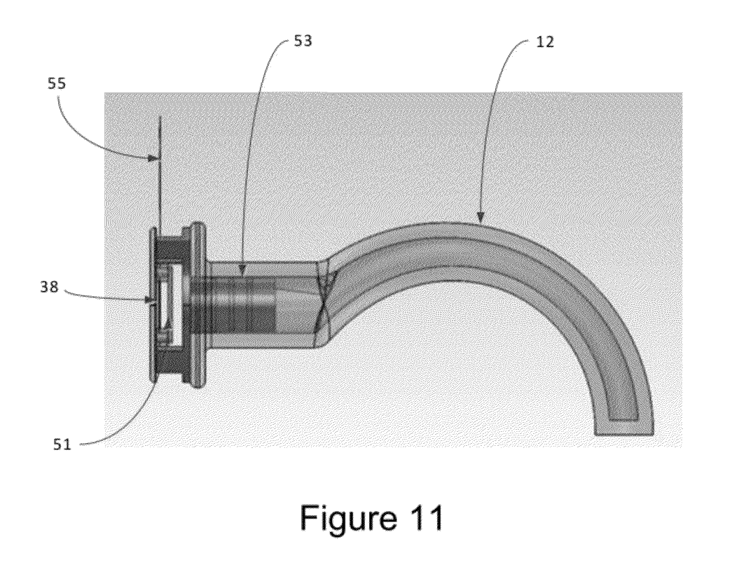

Advantageously, due to the positioning of the upper flange portion 30 and the reactive material 38 by glancing at the mouth of the patient, the reactive material becomes instantly visible. Additionally, while not obstructing the breathing of the patient, the portion of the airway that extends beyond the mouth (or nose) of the patient can be minimized. Furthermore, equipment covering the nose and mouth can be placed on the patient without inadvertently hitting, dislodging or otherwise harming the patient or the use of the airway. In addition, no changes are required to such standard equipment that is currently utilized. In certain embodiments, such as the embodiment shown in FIG. 11, a protective shield 51 may be positioned between the lower flange portion and the reactive material. This shield is configured to protect the reactive material from body fluids that may inadvertently be directed thereat. The protective shield may comprise a plurality of openings, or may comprise a porous or air permeable member. In other embodiments, such a configuration may be omitted altogether.

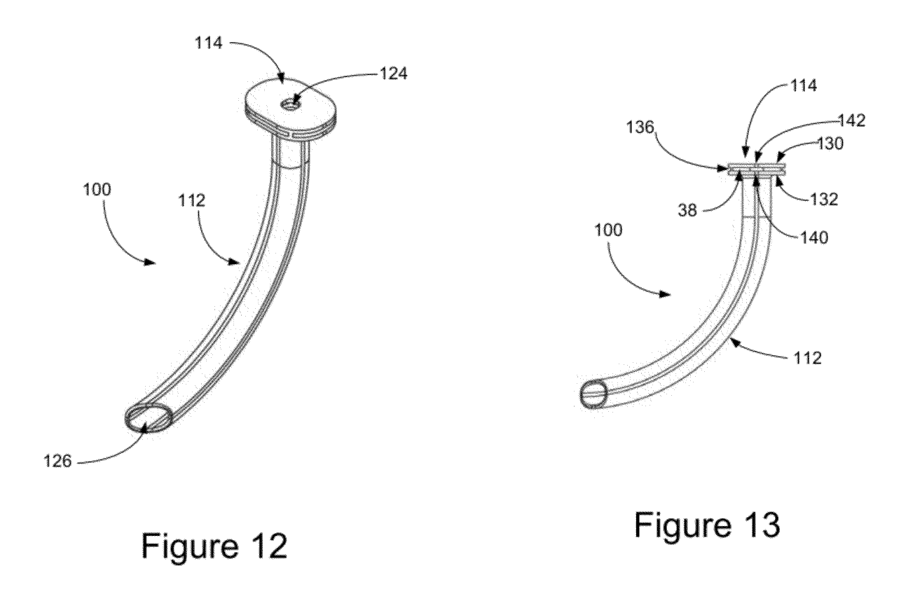

The same system can be applied to a nasopharyngeal airway as well. With reference to FIGS. 12 and 13, a nasopharyngeal airway is shown generally at 100. As with the oropharyngeal opening, the nasopharyngeal airway includes a passageway body 112 and a flange assembly 114. The passageway body has a proximal opening 124 and a distal opening 126. Typically, such airways tend to be more flexible in nature and typically have a circular, tube-like configuration. Of course, the disclosure is not limited to any particular dimensional configuration of the passageway body.

The flange assembly 114 includes upper flange portion 130 and lower flange portion 132. These flange portions are substantially parallel to each other, and may each have a slightly conical configuration so that they properly seat against the nasal passageway, and can accommodate a number of differently sized nasal passageways. Of course, other configurations are likewise contemplated. The upper flange portion 130 includes primary opening 142, and the lower flange portion includes opening 140.

The two flange portions are spaced apart from each other so as to define at least one channel therebetween. The two passageways are maintained in a spaced apart orientation by spacers 136 which may extend radially outward from the openings 140, 142. In other embodiments, they may extend only partially between the two, and they may also extend in different configurations and patterns.

As with the oropharyngeal airway, reactive material 38 can be integrally molded with the flange portions or can be applied as a coating over portions of the flange portions. The operation of the nasopharyngeal is much the same as the oropharyngeal airway, with the exception that breathing is done through the nose rather than the mouth. Also, as with the oropharyngeal airway, it is desirable to have the upper flange portion positioned proximate the lower flange portion so that the two are spaced apart less than one inch and more preferably less than one half inch. It is desirable that they are as close as possible, without undesirably obstructing the respiration of the patient.

The foregoing description merely explains and illustrates the disclosure and the disclosure is not limited thereto except insofar as the appended claims are so limited, as those skilled in the art who have the disclosure before them will be able to make modifications without departing from the scope of the disclosure.

What is claimed is:

1. An oropharyngeal airway configured for placement within a mouth of a patient to create a passageway between a mouth of a patient and the posterior pharyngeal wall comprising:
a passageway body having a proximal end and a distal end, the passageway body defining a passageway extending therethrough with an opening at the proximal end and an opening at the distal end;
a flange assembly positioned at the proximal end of the passageway body, the flange assembly configured to reside proximate a mouth of a patient when in use, and, including an upper flange portion and a lower flange portion, the upper and lower flange portions being spaced apart from each other to define a passageway therebetween which is in fluid communication with the proximal end of the passageway body, so that at least a portion of the air extending through the passageway body is directed through the passageway defined by the spaced apart flange portions, and
a reactive material cooperating with the flange assembly, the reactive material in communication with the passageway defined by the spaced apart flange portions, the reactive material providing a qualitative visual indication as to whether the patient is effectively ventilating.

2. The oropharyngeal airway of claim 1 wherein the reactive material is positioned on a lower surface of the upper flange portion so that it lies directly across from the opening of the passageway at the proximal end of the passageway body, and visible through the upper flange portion.

3. The oropharyngeal airway of claim 2 wherein at least a portion of the upper flange portion is substantially perpendicular to an axis defined by the passageway of the passageway body near the proximal end.

4. The oropharyngeal airway of claim 3 wherein the upper flange portion is substantially parallel to the lower flange portion and spaced apart less than one inch therefrom, and more preferably less than one half inch therefrom.

5. The oropharyngeal airway of claim 2 wherein an indicator sticker is positioned on the upper flange portion proximate the reactive material, to, in turn, allow for comparison thereto.

6. The oropharyngeal airway of claim 1 wherein the area of the passageway defined by the upper and lower flange portions is at least 80% the area of the passageway at the opening at the proximal end of the passageway body, so as to minimize any obstruction to the flow of air.

7. The oropharyngeal airway of claim 6 wherein the upper flange further includes an opening extending therethrough, the opening positioned above the opening at the proximal end of the passageway body and being sized so as to be smaller than the opening at the proximal end of the passageway body.

8. The oropharyngeal airway of claim 1 wherein the upper flange portion and the lower flange portion are coupled to each other through a plurality of spacers that, in turn, define a spacing of the passageway therebetween.

9. The oropharyngeal airway of claim 8 wherein the upper flange, the lower flange and the plurality of spacers are integrally molded with each other.

10. The oropharyngeal airway of claim 1 further comprising a bite protector inserted into the opening of the passageway body at the proximal end thereof, the upper flange portion being coupled thereto, whereupon insertion of the bite protector and coupling of the upper flange thereto, the upper flange portion is in operable position overlying the lower flange portion, while being spaced therefrom.

11. The oropharyngeal airway of claim 10 wherein the bite protector is integrally molded with the upper flange portion.

12. The oropharyngeal airway of claim 1 wherein the upper flange portion defines a footprint and the lower flange portion defines a footprint, the upper flange portion footprint remains within the confines of the footprint of the lower flange portion.

13. The oropharyngeal airway of claim 1 comprising one of a Berman type and a Guedel type airway.

14. An assembly configured for use with an oropharyngeal airway having a passageway body configured for placement within a mouth of a patient to create a passageway between a mouth of a patient and the posterior pharyngeal wall comprising:
a flange assembly couplable to an oropharyngeal airway, the flange assembly configured to reside proximate a mouth of a patient when an oropharyngeal airway is in operable position, and, including an upper flange portion being spaced apart from a proximal end of a passageway body of an airway to define a passageway therebetween, so that at least a portion of the air extending through a passageway body is directed through the passageway defined by the spaced apart flange portion, and
a reactive material cooperating with the flange assembly, the reactive material in communication with the passageway defined by the spaced apart flange portion, the reactive material providing a qualitative visual indication as to whether the patient is effectively ventilating.

15. The assembly of 14 further comprising a lower flange portion, the lower flange portion being spaced apart from the upper flange portion and configured to be positioned proximate a proximal end of a passageway body when the bite protector is properly positioned.

16. A nasopharyngeal airway configured for placement through a nostril of a patient to create a passageway between a nostril of a patient and the posterior pharyngeal wall comprising:
a passageway body having a proximal end and a distal end, the passageway body defining a passageway extending therethrough with an opening at the proximal end and an opening at the distal end;
a flange assembly positioned at the proximal end of the passageway body, the flange assembly configured to reside proximate a nostril opening of a patient when in use, and, including an upper flange portion and a lower flange portion, the upper and lower flange portions being spaced apart from each other to define a passageway therebetween which is in fluid communication with the proximal end of the passageway body, so that at least a portion of the air extending through the passageway body is directed through the passageway defined by the spaced apart flange portions, and
a reactive material cooperating with the flange assembly, the reactive material in communication with the passageway defined by the spaced apart flange portions, the reactive material providing a qualitative visual indication as to whether the patient is effectively ventilating.

17. The nasopharyngeal airway of claim 16 wherein the reactive material is positioned on a lower surface of the upper flange portion so that it lies directly across from the opening of the passageway at the proximal end of the passageway body, and visible through the upper flange portion.

18. The nasopharyngeal airway of claim 17 wherein at least a portion of the upper flange portion is substantially perpendicular to an axis defined by the passageway of the passageway body near the proximal end.

19. The nasopharyngeal airway of claim 18 wherein the upper flange portion is substantially parallel to the lower flange portion and spaced apart less than one inch therefrom.

20. The nasopharyngeal airway of claim 16 wherein the upper flange portion and the lower flange portion are coupled to each other through a plurality of spacers that, in turn, define the passageway therebetween.
